(19) United States
(12) Patent Application Publication
(10) Pub. No.: US 2016/0367462 A1
(43) Pub. Date: Dec. 22, 2016

(54) METHOD OF TREATING KERATIN MATTER BY FORMING AN IONIC LIQUID

(71) Applicant: L'OREAL, Paris (FR)

(72) Inventor: Henri SAMAIN, Bievres (FR)

(21) Appl. No.: 15/249,921

(22) Filed: Aug. 29, 2016

Related U.S. Application Data
(63) Continuation of application No. 14/896,419.

(30) Foreign Application Priority Data
Jun. 7, 2013 (FR) 1355252

(51) Int. Cl. A61K 8/49; A45D 2/00; A45D 1/00; A61K 8/24

(52) U.S. Cl. CPC A61K 8/4946; A61K 8/24; A45D 2/001; A45D 1/00; A61K 2800/884; A61K 2800/592; A61K 2800/43

(57) ABSTRACT

The subject of the invention is a method for treating keratin materials, such as keratin fibers, which comprises i) application of a composition comprising at least one first, water-soluble hydrophilic ionic liquid A⁺X⁻ comprising an organic cation A⁺ and ii) application of a composition comprising a soluble hydrophilic salt B⁻Y⁺ comprising an anion B⁻, the anion B⁻ being such that it forms a second, hydrophobic ionic liquid A⁺B⁻ by ion exchange with the first ionic liquid A⁺X⁻.

This method for treating keratin materials makes it possible to improve the penetration of cosmetic active agents while retaining a cosmetic feel.

The present invention relates to a method for the cosmetic treatment of keratin materials, especially the skin and the hair, to provide a long-lasting effect to the keratin materials.

In the field of cosmetics, it is very often sought to improve the penetration of cosmetic active agents into the skin or into the hair, so as to obtain improved cosmetic results. One of the ways which makes it possible to improve the penetration of active agents is to combine them with fatty compounds, often in large amounts. This approach, which may be effective, nonetheless has the drawback of leaving a greasy feel on the skin or the hair, which is often unsightly.

Another way to improve the penetration of active agents is the use of surfactants. The fatty substances are then compatible with an aqueous medium. With this approach, a tacky feel which is not very cosmetic is often obtained.

Moreover, these two approaches are not entirely satisfactory in terms of the degree of penetration into the skin or the hair.

It is therefore important to be able to develop systems which enable a better penetration of active agents into the keratin materials, while retaining a pleasant cosmetic feel for the user, in particular a non-greasy, non-shiny and/or non-tacky feel.

Thus, depending on the active agents chosen, it is possible to envision improving the appearance of the skin or the hair for example to repair or modify the state of the keratin materials when they are damaged, to strengthen the hair, to improve the persistence of dyeing, to give volume to the hair, to straighten or curl the hair, etc.

The object of the invention is to provide a method for treating keratin materials which makes it possible to improve the penetration of cosmetic active agents while retaining a cosmetic feel.

Thus, the subject of the invention is a method for treating keratin materials which comprises i) application of a composition comprising at least one first, water-soluble ionic liquid A⁺X⁻ comprising an organic cation A⁺ and ii) application of a composition comprising a water-soluble salt B⁻Y⁺ comprising an anion B⁻, the anion B⁻ being such that it forms a second, hydrophobic ionic liquid A⁺B⁻ by ion exchange with the first ionic liquid A⁺X⁻.

The method of the invention may especially be applied to keratin materials such as keratin fibers, for example the hair or the skin.

In the method of the invention, the steps i) and ii) may be carried out in any order.

Within the context of the present invention, the term “ionic liquid” means a salt of an organic molecule, said salt having a melting point of less than or equal to 150° C., preferably less than 100° C. Preferably, the salt remains liquid up to 300° C., and more preferably the salt is liquid at room temperature, that is to say at a melting point of less than or equal to 50° C. and greater than 0° C.

The melting point is measured by differential calorimetric analysis, with a temperature increase rate of 10° C./minute, the melting point then being at a temperature corresponding to the top of the endothermic melting peak obtained during the measurement.

According to the invention, the term “hydrophobic ionic liquid” means an ionic liquid having a solubility in water at room temperature (25° C.) of less than 5%, preferably less than 1% by weight, or even less than 0.5%. Within the context of the invention, the hydrophobic character of the ionic liquid is such that there is phase separation in water.

Within the context of the invention, the term “water-soluble” means a salt or an ionic liquid which has a solubility in water at 25° C. of greater than 1%, preferably greater than 5%, more preferably greater than 10%; that is to say, which forms at this concentration a macroscopically homogeneous, transparent and isotropic medium.

The ionic liquids used according to the invention have an organic or inorganic, preferably organic, cation A⁺ chosen from the imidazolium, pyrazolium, pyridinium, pyrimidinium, tetra(C₁-C₆)alkylphosphonium, tetra(C₁-C₆)alkylammonium, guanidinium, cholinium, pyrrolidinium, uronium, thiouronium and isothiouronium cations.

By way of example, the imidazolium cations may correspond to the following formula:

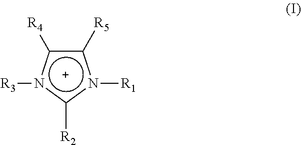

in which R₁ represents an alkyl group comprising from 1 to 15 carbon atoms, preferably from 1 to 6 carbon atoms, optionally substituted by one or more C₆-C₃₀ aryl groups, thiol groups or hydroxy groups or interrupted by one or more oxygen or sulfur atoms or by one or more NR′ groups,
R₂, R₃, R₄, R′ and R₅, which are identical or different, each represent a hydrogen atom or an alkyl group comprising from 1 to 5 carbon atoms, preferably from 1 to 4 carbon atoms, or a C₆-C₃₀ aryl group, optionally substituted by one or more C₁-C₄ alkyl groups.

Preferably, R₁ and R₃, which are identical or different, each represent an alkyl group comprising from 1 to 4 carbon atoms.

The pyrazolium cations may correspond to one of the following formulae:

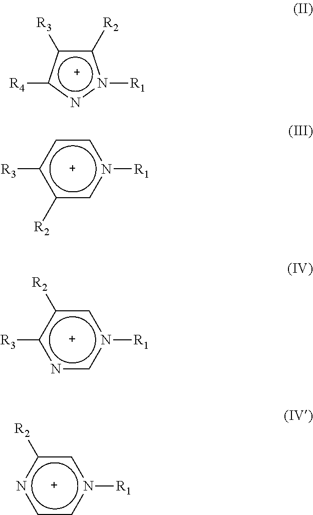

in which R₁, R₂, R₃ and R′, which are identical or different, are as defined above.

The tetra(C₁-C₆)alkylphosphonium cations may correspond to the following formula:

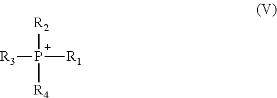

in which R₁, R₂ and R₃ and R₄, which are identical or different, represent an alkyl group comprising from 1 to 15 carbon atoms and preferably from 1 to 6 carbon atoms, optionally substituted by one or more C₆-C₃₀ aryl groups, thiol groups or hydroxy groups or interrupted by one or more oxygen or sulfur atoms or by one or more NR′ groups,
R′ represents a hydrogen atom or an alkyl group comprising from 1 to 5 carbon atoms and preferably from 1 to 4 carbon atoms, or a C₆-C₃₀ aryl group, optionally substituted by one or more C₁-C₄ alkyl groups.

The tetra(C₁-C₆)alkylammonium cations correspond in particular to the following formula:

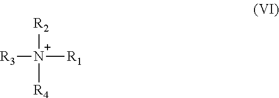

in which R₁, R₂, R₃ and R₄, which are identical or different, represent an alkyl group comprising from 1 to 6 carbon atoms and preferably from 1 to 4 carbon atoms, optionally substituted by one or more C₆-C₃₀ aryl groups, thiol groups or hydroxy groups or interrupted by one or more oxygen or sulfur atoms or by one or more NR′ groups,
R′ represents a hydrogen atom or an alkyl group comprising from 1 to 5 carbon atoms and preferably from 1 to 4 carbon atoms, or a C₆-C₃₀ aryl group, optionally substituted by one or more C₁-C₄ alkyl groups. Preferably, the alkyl group of R₁, R₂, R₃ and R₄ is not substituted.

The guanidinium cations may correspond to the following formula:

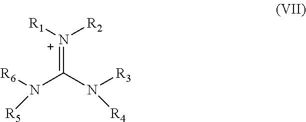

in which R₁ to R₆, which are identical or different, represent a hydrogen atom or an alkyl group comprising from 1 to 15 carbon atoms, preferably from 1 to 6 carbon atoms, optionally substituted by one or more C₆-C₃₀ aryl groups, thiol groups or hydroxy groups or interrupted by one or more oxygen or sulfur atoms or by one or more NR′ groups,

R′ represents a hydrogen atom or an alkyl group comprising from 1 to 5 carbon atoms and preferably from 1 to 4 carbon atoms, or a C₆-C₃₀ aryl group, optionally substituted by one or more C₁-C₄ alkyl groups.

The cholinium cations may correspond to the following formula:

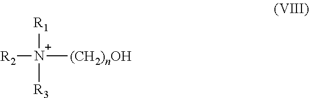

in which R₁, R₂ and R₃, which are identical or different, each represent an alkyl group comprising from 1 to 15 carbon atoms, preferably from 1 to 6 carbon atoms, optionally substituted by one or more C₆-C₃₀ aryl groups, thiol groups or hydroxy groups or interrupted by one or more oxygen or sulfur atoms or by one or more NR′ groups,
R′ represents a hydrogen atom or an alkyl group comprising from 1 to 5 carbon atoms and preferably from 1 to 4 carbon atoms, or a C₆-C₃₀ aryl group, optionally substituted by one or more C₁-C₄ alkyl groups,
n is an integer ranging from 1 to 15, preferably from 1 to 12.

The pyrrolidinium cations may correspond to the formula:

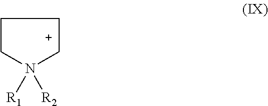

in which R₁ represents an alkyl group comprising from 1 to 15 carbon atoms, preferably from 1 to 6 carbon atoms, optionally substituted by one or more C₆-C₃₀ aryl groups, thiol groups or hydroxy groups or interrupted by one or more oxygen or sulfur atoms or by one or more NR′ groups,
R₂ and R′ represent, independently of one another, a hydrogen atom or an alkyl group comprising from 1 to 5 carbon atoms and preferably from 1 to 4 carbon atoms, or a C₆-C₃₀ aryl group, optionally substituted by one or more C₁-C₄ alkyl groups.

The isouronium cations may correspond to the following formula:

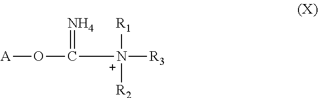

in which R₁ to R₄ and A, which are identical or different, each represent a C₁-C₁₅, preferably C₁-C₆ alkyl group optionally substituted by one or more C₆-C₃₀ aryl groups, thiol groups or hydroxy groups or interrupted by one or more oxygen or sulfur atoms or by one or more NR′ groups,
R′ represents a hydrogen atom or an alkyl group comprising from 1 to 5 carbon atoms and preferably from 1 to 4 carbon atoms, or a C₆-C₃₀ aryl group, optionally substituted by one or more C₁-C₄ alkyl groups.

The isothiouronium cations may correspond to the following formula:

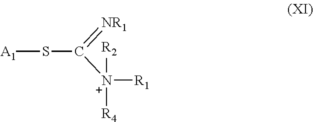

in which R₁ to R₄ and A₁, which are identical or different, each represent a C₁-C₁₅, preferably C₁-C₆ alkyl group optionally substituted by one or more C₆-C₃₀ aryl groups, thiol groups or hydroxy groups or interrupted by one or more oxygen or sulfur atoms or by one or more NR′ groups,
R′ represents a hydrogen atom or an alkyl group comprising from 1 to 5 carbon atoms and preferably from 1 to 4 carbon atoms, or a C₆-C₃₀ aryl group, optionally substituted by one or more C₁-C₄ alkyl groups.

The uronium cations may correspond to the following formula:

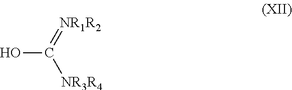

in which R₁ to R₄, which are identical or different, each represent a C₁-C₁₅, preferably C₁-C₆ alkyl group optionally substituted by one or more C₆-C₃₀ aryl groups, thiol groups or hydroxy groups or interrupted by one or more oxygen or sulfur atoms or by one or more NR′ groups,
R′ represents a hydrogen atom or an alkyl group comprising from 1 to 5 carbon atoms and preferably from 1 to 4 carbon atoms, or a C₆-C₃₀ aryl group, optionally substituted by one or more C₁-C₄ alkyl groups.

The cations A⁺ may be constituted of cosmetic active agents comprising a quaternary ammonium radical. By way of example, mention may be made of sunscreens of the merocyanine type, azo dyes and cationic dyes.

Preferably, the cation A⁺ of the ionic liquids used according to the invention is chosen from ammonium, imidazolium and cholinium cations. According to one embodiment, these cations may be substituted by cosmetic active agents such as described above.

Among the cations which may be used, mention may be made of 1-methylimidazolium; 1-butylimidazolium; 3-methyl-1-tetradecylimidazolium; 1-hexadecyl-3-methylimidazolium, 1-phenylpropyl-3-methylimidazolium; 1-ethyl-2,3-dimethylimidazolium; 1-propyl-2,3-dimethylimidazolium; 1-butyl-2,3-dimethylimidazolium; 1-hexadecyl-2,3-dimethylimidazolium; N-ethylpyridinium; N-butyl-3-methylpyridinium; 4-methyl-N-butylpyridinium; N-butyl-3,4-dimethylpyridinium; 3-ethyl-N-butylpyridinium; N-hexylpyridinium; 3-methyl-N-hexylpyridinium; 4-methyl-N-hexylpyridinium; N-octylpyridinum; 3-methyl-N-octylpyridinium; 4-methyl-N-octylpyridinium; tetrabutylphosphonium; triisobutyl(methyl)phosphonium; benzyltriphenylphosphonium; ethyl(tributyl)phosphonium; methyl(tributyl)phosphonium; tetraoctylphosphonium; tetramethylammonium; 1,1-dimethylpyrrolidinium; 1-ethyl-1-methylpyrrolidinium; 1,1-dipropylpyrrolidinium; 1-butyl-1-methylpyrrolidinium; 1-butyl-1-ethylpyrrolidinium; 1,1-dibutylpyrrolidinium; 1-hexyl-1-methylpyrrolidinium; 1,1-dihexylpyrrolidinium; 1-octyl-1-methylpyrrolidinium; guanidinium; N,N,N′,N′-tetramethyl-N″-ethylguanidinium; N-pentamethyl-N-isopropylguanidinium; hexamethylguanidinium; O-methyl-N,N,N′,N′-tetramethylisouronium; S-ethyl-N, N, N′,N′-tetramethylisothiouronium; 1-butyl-3-ethylimidazolium.

According to the present invention, the cation Y⁺ may be chosen from the monocations, such as those of the alkali metals, amines and ammoniums or other nitrogenous derivatives such as guanidiniums.

The ionic liquids A⁺X⁻ or A⁺B⁻ have an inorganic or organic anion B⁻ or X⁻.

According to one particular embodiment, B⁻ is chosen such that A⁺B⁻ represents a water-soluble salt. B⁻ is especially chosen from weakly coordinating anions such as the polyhalogenatedalkyl or polyaryl anions, anions with a hydrophobic carbon-based chain, long chain alkyl sulfates, preferably of more than 5 carbon atoms, halophosphate ions, (di/tri)haloacetate ions, (di/tri)halomethane sulfonate ions and halosulfate ions, the halogen preferably being fluorine. By way of example of anions B⁻, mention may be made of hexafluorophosphate, tetrafluoroborate, the tetraarylborates, bistriflimide, the alky sulfates with chains > C5, such as octyl sulfate(C₈H₁₇OSO₃₋), the tris(pentafluoroethyl)trifluorophosphate ion; the triflate [TfO] ion (CF₃SO₂⁻), the nonaflate [NfO] ion (CF₃(CF₂)₃SO₂⁻), a bis(trifyl)amide [Tf₂N] ion ((CF₃SO₂)₂N⁻); the trifluoroacetate [TA] ion (CF₃CO₂³¹ ) and heptafluorobutanoate [HB] ion (CF₃(CF₂)₃CO₂⁻); the trifluoromethanesulfonate ion (CF₃SO₃⁻); and the dicyanamide or salicylate ions.

The anion X⁻ is generally chosen from chlorides and bromides, short-chain carboxylates, especially C₁-C₄ carboxylates, nitrates and anionic oxide compounds. By way of example, mention may be made of the chloride (Cl⁻) or bromide (Br⁻) ions; the tetrahaloaluminate ions such as tetrachloroaluminate (AlCl₄⁻); the tetrahalonickel ions such as tetrachloronickel (NiCl₄⁻), the perchlorate ion (ClO₄⁻), the nitrate ion (NO₃⁻); the nitrite ion (NO₂⁻) the sulfate ion (SO₄²⁻), the hydrogensulfate ion, the phosphate ion, and the ions derived from the phosphate ion, acetates and formates. It may also be chosen from the organic anions such as the methyl sulfate ion (CH₃SO₄⁻), dibutylphosphate, and citrate or lactate ions.

Nonlimitingly, the ionic liquids A⁺X⁻ used according to the invention may be chosen from the following compounds:
1-ethyl-3-methylimidazolium chloride,
1-ethyl-3-methylimidazolium bromide,
1-butyl-3-methylimidazolium chloride,
1-hexyl-3-methylimidazolium chloride,
1-methyl-3-octylimidazolium chloride,
1-decyl-3-methylimidazolium chloride,
1-decyl-3-methylimidazolium bromide,
1-dodecyl-3-methylimidazolium chloride,
1-methyl-3-tetradecylimidazolium chloride,
4-methyl-N-butylpyridinium chloride,
3-methyl-N-butylpyridinium chloride,
4-methyl-N-hexylpyridinium chloride,
1,3-dimethylimidazolium methyl sulfate,
1-methyl-3-butylimidazolium methyl sulfate,
1-ethyl-3-methylimidazolium acetate,
1-ethyl-3-methylimidazolium sulfate,
1-butylpyridinium bromide,
1-butylpyrimidinium trifluoromethanesulfonate,
1-hexylpyrimidinium trifluoromethanesulfonate,
1-ethyl-3-methylimidazolium trifluoroacetate,
trihexyltetradecylphosphonium chloride,
tributyltetradecylphosphonium chloride,
1-ethyl-3-methylimidazolium trifluoroacetate,
1-hexyl-2,3-dimethylimidazolium chloride,
1-ethyl-2,3-dimethylimidazolium chloride,
1-ethyl-3-methylimidazolium dicyanamide,
tetrabutylammonium hydroxide,
choline salicylate,
tributylmethylammonium methyl sulfate,
cholinium acetate,
tetraethylammonium acetate tetrahydrate,
triethylmethylammonium dibutylphosphate,
1-ethyl-3-methylimidazolium L-(+)-lactate,
and hydrates thereof.

Nonlimitingly, the salts B⁻Y⁺ used according to the invention may be chosen from the following compounds:
ammonium or alkali metal (Na⁺, etc.) hexafluorophosphate
ammonium or alkali metal tetrafluoroborate
ammonium or alkali metal bistriflimide
ammonium or alkali metal octyl sulfate.

According to a particular embodiment the ionic liquid A⁺B⁻ formed at the moment of application is liquid or pasty at room temperature.

By way of example of hydrophobic ionic liquid A⁺B⁻ mention may be made of 3-methyloctylimidazolium hexafluorophosphate, 3-methylbutylimidazolinium hexafluorophosphate, 1-ethyl-3-methylimidazolium hexafluorophosphate, 1-hexyl-3-methylimidazolium hexafluorophosphate, 1-decyl-3-methylimidazolium hexafluorophosphate, 1-dodecyl-3-methylimidazolium hexafluorophosphate, 1,3-dimethylimidazolium hexafluorophosphate, 4-methyl-N-butylpyridinium hexafluorophosphate, 3-methyl-N-butylpyridinium hexafluorophosphate, 4-methyl-N-hexylpyridinium hexafluorophosphate, 1-ethyl-3-methylimidazolium hexafluorophosphate, 1-butylpyridinium hexafluorophosphate, arylazodimethylimidazolium hexafluorophosphate and arylazomethylpyridinium hexafluorophosphate.

The ionic liquids generally represent from 1% to 100%, preferably from 10% to 100%, and better still from 40% to 100% by weight relative to the total weight of the composition of the invention.

The cosmetic composition used according to the invention may also comprise one or more compounds which are liquid at room temperature other than the ionic liquids used in the method of the invention.

The liquid compound is preferably a solvent and in particular a solvent chosen from water, aliphatic C₁-C₄ alcohols such as ethanol and isopropanol, organic solvents which are soluble or dispersible in water such as acetone, propylene carbonate, benzyl alcohol, derivatives of glycol ether, propylene glycol, polyols such as glycerol, and polyethylene glycols.

Preferably, the liquid compound is a polar solvent.

It has been observed that the method according to the invention makes it possible to obtain greater penetration of the ionic liquids into the keratin materials compared to the methods of the prior art which comprise the direct application of the lipophilic ionic liquid. Thus, with the method of the invention, a method for treating keratin materials with ionic liquids having better effectiveness is obtained. It is thus possible to envision, as a function of the ionic liquid formed in situ, greater effectiveness, for example during the application to keratin fibers, for strengthening and/or repairing the hair, dyeing the hair, conditioning the hair in particular to provide or improve disentangling, smoothing, combability, manageability, the softness of the head of hair, in a longer-lasting way. On the skin, it is possible to envision better, longer-lasting moisturizing of the skin.

The method may also comprise at least one step of rinsing.

When the method of the invention is applied to keratin fibers, in particular the hair, it may comprise a step of heat treatment. Thus, the application of the composition to the hair may optionally be followed by a step of rinsing and/or a step of heat treatment, for example at a temperature of between 150° C. and 250° C.

The method of the invention may comprise an intermediate rinsing step between the application of the ionic liquid(s) A⁺X⁻ and the salt B⁻Y⁺. It may also comprise a final rinse. According to one particular embodiment, the method of the invention does not comprise a final rinse.

The compositions of use in the method of the invention may also comprise at least one common cosmetic ingredient, chosen especially from propellants; oils; solid fatty substances and especially C₈-C₄₀ esters; C₈-C₄₀ acids; C₈-C₄₀ alcohols; nonionic surfactants; cationic surfactants; anionic surfactants; amphoteric surfactants; zwitterionic surfactants; sunscreens; moisturizers; antidandruff agents; antioxidants; chelating agents; nacreous agents and opacifiers; plasticizers or coalescers; hydroxy acids; fillers; silicones and in particular polydimethylsiloxanes; polymeric or non-polymeric thickeners; gelling agents; emulsifiers; polymers, in particular conditioning or hairstyling polymers; fragrances; basifying agents or acidifying agents; silanes; crosslinking agents. The composition can, of course, comprise several cosmetic ingredients appearing in the above list.

Depending on their nature and the purpose of the compositions, the common cosmetic ingredients may be present in usual amounts which can be easily determined by those skilled in the art and which can be, for each ingredient, between 0.01% and 80% by weight.

### EXAMPLE

### Example 1

The following two compositions are produced:

Composition a:
13.5% 3-methyloctylimidazolium chloride (CAS 64697-40-1)
86.5% water

Composition b:
10% ammonium hexafluorophosphate (CAS 16941-11-0)
90% water

The two compositions are perfectly clear and soluble.

When the two compositions are mixed, an ionic liquid based on 3-methyloctylimidazolium hexafluorophosphate is obtained, which is not soluble in water. Thus, immediately after mixing, a 2-phase mixture is obtained.

In accordance with our invention, first composition a is applied to the hair or the skin, then 5 min later, composition b is applied.

By way of comparison, a composition c containing 10% 3-methyloctylimidazolium hexafluorophosphate (ionic liquid) and 90% water is applied.

The successive application of composition a then b gives a much less greasy result than applying composition c to the skin and the hair.

After rinsing and washing the hair or skin, much soft hair or skin is obtained than with the method of the invention compared to application of composition c.

### Example 2

The following composition is produced:

Composition d:
10% 3-methylbutylimidazolium chloride (CAS 64697-40-1)
90% water

Composition b from example 1 is also produced.

The two compositions are perfectly clear and soluble.

When the two compositions are mixed, an ionic liquid based on 3-methylbutylimidazolium hexafluorophosphate is obtained, which is not soluble in water. Thus, immediately after mixing, a 2-phase mixture is obtained.

In accordance with our invention, first composition d is applied to the hair or the skin, then 5 min later, composition b is applied.

By way of comparison, a composition e containing 10% 3-methylbutylimidazolium hexafluorophosphate (ionic liquid) and 90% water is applied.

The successive application of composition a then b gives a much less greasy result than applying composition e to the skin and the hair.

After rinsing and washing the hair or skin, much soft hair or skin is obtained than with the method of the invention compared to application of composition c.

### Example 3

A second test is carried out, using a dye as first ionic liquid A⁺X⁻.

Two compositions are produced:

| | |
|---|---|
| Composition f | |
| Basic Red 51 dye | 2 millimol (0.56 g) |
| (aminoarylazodimethylimidazolium | |
| chloride of molar mass 279.5 mol/g, | |
| CAS 77061-58-6) supplied by Ciba | |
| Water qs | 100 g |
| Composition g | |
| Ammonium hexafluorophosphate | 2 millimol (0.326 g) |
| Water qs | 100 g |

Three experiments are carried out:

Experiment 1: composition f is applied, left on for 10 min, then composition g is applied which is left on for poser 5 min, then rinsing is carried out.

Experiment 2: composition f is applied, left on for 10 min, then rinsing is carried out.

Experiment 3: Composition g is applied, left on for 5 min, then f is applied, left on for 10 min, then rinsing is carried out.

At the moment of rinsing, it is observed that the locks from experiments 1 and 3 run much less than the lock from experiment 2.

After drying the locks, the locks from experiments 1 and 3 have a much darker color.

1- A method for treating keratin materials which comprises i) application of a composition comprising at least one first, water-soluble ionic liquid A⁺X⁻ comprising an organic cation A⁺ and ii) application of a composition comprising a water-soluble salt B⁻Y⁺ comprising an anion B⁻, the anion B⁻ being such that it forms a second, hydrophobic ionic liquid A⁺B⁻ by ion exchange with the first ionic liquid A⁺X⁻.

2- The method as claimed in claim 1, wherein the first and the second ionic liquid has a melting point of less than or equal to 50° C. and greater than 0° C.

3- The method as claimed in claim 1 or 2, wherein the cation A⁺ is an organic chosen from the imidazolium, pyrazolium, pyridinium, pyrimidinium, tetra(C₁-C₆) alkylphosphonium, tetra(C₁-C₆)alkylammonium, guanidinium, cholinium, pyrrolidinium, uronium, thiouronium and isothiouronium cation.

4- The method as claimed in claim 1 or 2, wherein the cation Y⁺ is chosen from the monocations, such as those of the alkali metals, amines and ammoniums or other nitrogenous derivatives such as guanidiniums.

5- The method as claimed in claim 1 or 2, wherein the anions B⁻ are chosen from weakly coordinating anions chosen from the polyhalogenatedalkyl or polyaryl anions, anions with a hydrophobic carbon-based chain, long chain alkyl sulfates of more than 5 carbon atoms, halophosphate ions, (di/tri)haloacetate ions, (di/tri)halomethane sulfonate ions and halosulfate ions, the halides being fluoride.

6- The method as claimed in claim 1 or 2, wherein the anions X⁻ are chosen from bromides, chlorides, C1-C4 short-chain carboxylates, nitrates, anionic oxide compounds, tetrahaloaluminate ions, tetrahalonickel ions, the perchlorate ion (ClO₄⁻), the nitrate ion (NO₃⁻); the nitrite ion (NO₂⁻); the sulfate ion (SO₄²⁻), the hydrogensulfate ion, the phosphate ion, acetates and formates, and citrate or lactate ions.

7- The method as claimed in any one of the preceding claims, wherein A⁺X⁻ is chosen from the following compounds:
1-ethyl-3-methylimidazolium chloride,
1-ethyl-3-methylimidazolium bromide,
1-butyl-3-methylimidazolium chloride,
1-hexyl-3-methylimidazolium chloride,
1-methyl-3-octylimidazolium chloride,
1-decyl-3-methylimidazolium chloride,
1-decyl-3-methylimidazolium bromide,
1-dodecyl-3-methylimidazolium chloride,
1-methyl-3-tetradecylimidazolium chloride,
4-methyl-Nbutyl-pyridinium chloride,
3-methyl-N-butylpyridinium chloride,
4-methyl-N-hexylpyridinium chloride,
1,3-dimethylimidazolium methyl sulfate,
1-methyl-3-butylimidazolium methyl sulfate,
1-ethyl-3-methylimidazolium acetate,
1-ethyl-3-methylimidazolium sulfate,
1-butylpyridinium bromide,
1-butylpyrimidinium trifluoromethanesulfonate,
1-hexylpyrimidinium trifluoromethanesulfonate,
1-ethyl-3-methylimidazolium trifluoroacetate,
trihexyltetradecylphosphonium chloride,
tributyltetradecylphosphonium chloride,
1-ethyl-3-methylimidazolium trifluoroacetate,
1-hexyl-2,3-dimethylimidazolium chloride,
1-ethyl-2,3-dimethylimidazolium chloride,
1-ethyl-3-methylimidazolium dicyanamide,
tetrabutylammonium hydroxide
choline salicylate,
tributylmethylammonium methyl sulfate,
cholinium acetate
tetraethylammonium acetate tetrahydrate,
triethylmethylammonium dibutylphosphate,
1-ethyl-3-methylimidazolium L-(+)-lactate,
and hydrates thereof.

8- The method as claimed in any one of the preceding claims, wherein B⁻Y⁻ is chosen from the following compounds:
ammonium or alkali metal hexafluorophosphate
ammonium or alkali metal tetrafluoroborate
ammonium or alkali metal bistriflimide
ammonium or alkali metal octyl sulfate.

9- The method as claimed in any one of the preceding claims, wherein A⁺B⁻ is chosen from 3-methyloctylimidazolium hexafluorophosphate, 3-methylbutylimidazolinium hexafluorophosphate, 1-ethyl-3-methylimidazolium hexafluorophosphate, 1-hexyl-3-methylimidazolium hexafluorophosphate, 1-decyl-3-methylimidazolium hexafluorophosphate, 1-dodecyl-3-methylimidazolium hexafluorophosphate, 1,3-dimethylimidazolium hexafluorophosphate, 4-methyl-N-butylpyridinium hexafluorophosphate, 3-methyl-N-butylpyridinium hexafluorophosphate, 4-methyl-N-hexylpyridinium hexafluorophosphate, 1-ethyl-3-methylimidazolium hexafluorophosphate, 1-butylpyridinium hexafluorophosphate, arylazodimethylimidazolium hexafluorophosphate and arylazomethylpyridinium hexafluorophosphate.

10- The method as claimed in any one of the preceding claims, wherein i) then ii) are applied in succession.

11- The method as claimed in any one of the preceding claims, comprising a step of rinsing and/or a step of heat treatment, especially with a straightening or curling iron, at a temperature of between 150° C. and 250° C.
